(19) United States
(12) Patent Application Publication
(10) Pub. No.: US 2026/0334938 A1
(43) Pub. Date: Oct. 29, 2026

(54) IMPLANTATION APPARATUS FOR LATERAL ROW ANCHOR AND IMPLANTATION SYSTEM FOR LATERAL ROW ANCHOR

(71) Applicant: STAR SPORTS MEDICINE CO., LTD., Beijing (CN)

(72) Inventors: Wenxing Dong, Beijing (CN); Xiongbin Jie, Beijing (CN); Zhixu Xu, Beijing (CN)

(21) Appl. No.: 19/418,921

(22) Filed: Dec. 13, 2025

Related U.S. Application Data
(63) Continuation of application No. PCT/CN2025/124015, filed on Sep. 25, 2025.

(30) Foreign Application Priority Data
Apr. 25, 2025 (CN) 202510526377.9

(51) Int. Cl. A61B 17/04

(52) U.S. Cl. CPC A61B 17/0401; A61B 2017/0409; A61B 2017/0464

(57) ABSTRACT

The present application relates to the technical field of medical instruments and discloses an implantation apparatus for a lateral row anchor and an implantation system for a lateral row anchor. The implantation apparatus for a lateral row anchor includes a snap head, an implantation rod, and a handle. The snap head has a connection channel; the implantation rod passes through the connection channel, the implantation rod can be inserted to an anchor box and is connected to a lateral row anchor in the anchor box, and the implantation rod is configured to eject the lateral row anchor threaded with a suture out of the anchor box; the handle is provided at the other end of the implantation rod and configured to drive the implantation rod to rotate, the implantation rod has a thread segment, and the snap head is provided with an abutting section.

### CROSS-REFERENCE TO RELATED APPLICATION

This application is a continuation of International Application No. PCT/CN2025/124015, filed on Sep. 25, 2025, which claims priority to Chinese Patent Application CN202510526377.9 filed on Apr. 25, 2025, both of which are hereby incorporated by reference in their entireties.

### TECHNICAL FIELD

The present application relates to the technical field of medical instruments, and particularly, to an implantation apparatus for a lateral row anchor and an implantation system for a lateral row anchor.

### BACKGROUND

In the field of orthopedics and sports medicine, an anchor is widely used in surgery for fixing soft tissue (such as a tendon and a ligament) of joints such a shoulder, a knee, and an ankle to bone tissue as a key implantable medical instrument. The anchor includes a medial row anchor and a lateral row anchor. The medial row anchor itself carries a suture, and the lateral row anchor itself does not carry a suture. After implantation of the medial row anchor is completed, the suture of the medial row anchor is introduced to the lateral row anchor, and then the lateral row anchor is implanted into a designated position to complete a suture fixation.

In the related art, the anchor and an implantation component are used in a complete set, one lateral row anchor is implanted each time, and the implantation component is discarded, causing that the implantation component cannot be reused and leading to the waste of medical resources. Although there are implantation systems that load the anchor using an anchor storage apparatus and work with the implantation component for implantation in the related art, the implantation systems are all applicable for the medial row anchor itself carrying the suture.

### SUMMARY

One of the objects of the present application is to provide an implantation apparatus for a lateral row anchor and an implantation system for a lateral row anchor to solve the problem of waste of medical resources caused by not reusing implantation components in the related art.

To achieve this object, the present application provides the following technical solutions.

In a first aspect, the present application provides an implantation apparatus for a lateral row anchor including:
a snap head having a connection channel;
an implantation rod passing through the connection channel, wherein one end of the implantation rod passing through the connection channel is capable of being inserted to an anchor box and inserted and connected to a lateral row anchor in the anchor box and is configured to implant the lateral row anchor threaded with a suture into a designated position after the lateral row anchor threaded with the suture is ejected out of the anchor box;
a handle provided at the other end of the implantation rod and configured to drive the implantation rod to rotate, wherein the implantation rod has a thread segment, and the snap head is provided with an abutting section, the abutting section is snapped into a thread of the thread segment to drive, under a condition that the implantation rod rotates, the implantation rod to move along an axial direction relative to the snap head and implant the lateral row anchor into the designated position; and

Optionally, a plurality of the abutting sections are provided at intervals.

Optionally, an end of the implantation rod away from the handle has a connection segment capable of being inserted to the lateral row anchor, and a head end of the connection segment has a connection protrusion for closely abutting against an inner wall of the lateral row anchor.

Optionally, the connection protrusion is an arc in shape, and an inner side of a head of the lateral row anchor has a step hole closely abutting against the connection protrusion.

Optionally, a section of the connection segment is a polygon in shape, and a side wall of the connection segment is fitted to an inner side wall of the lateral row anchor.

Optionally, the implantation rod is inserted and connected to the handle, and the handle is provided with a locking section abutting against an end of the implantation rod inserted and connected to the handle.

Optionally, the implantation apparatus for a lateral row anchor further includes:
a protective sleeve sleeved on the thread segment of the implantation rod, wherein the abutting section passes through the protective sleeve and is snapped into the thread of the thread segment.

Optionally, the snap head has a fixation section abutting against an outer side wall of the protective sleeve.

Optionally, the protective sleeve has a guide block, the anchor box has an insertion channel to which the protective sleeve is inserted, and the insertion channel has a guide slot on which the guide block slides.

Optionally, the snap head has an accommodation cavity for accommodating the anchor box, and a cavity wall of the accommodation cavity is provided with a locking piece for snapping into the anchor box to lock the anchor box in the accommodation cavity.

In a second aspect, the present application further provides an implantation system for a lateral row anchor including:
any one of the implantation apparatuses of the first aspect;
an anchor box storing a lateral row anchor and inserted and connected to the implantation apparatus for a lateral row anchor; and
a pull suture component for threading a suture for the lateral row anchor in the anchor box.

Optionally, the anchor box includes:
box body, wherein an end of the box body has an insertion channel to which the implantation rod is partially inserted, and the other end has an anchor-discharging channel for the lateral row anchor to slide out; and
a clamp anchor component provided in the box body and correspondingly communicated with the insertion channel, wherein the lateral row anchor is clamped in the clamp anchor component.

Optionally, the clamp anchor component includes two snap anchor housings opposite to each other, the lateral row anchor is stored between the two snap anchor housings, the snap anchor housings are actively connected to the box body, one end of the snap anchor housings away from the insertion channel is sleeved with an elastic sleeve, an inner side and one end away from the insertion channel of the snap anchor housings have a snap inclined surface abutting against a head end of the lateral row anchor, and the end of the two snap anchor housings sleeved with the elastic sleeve is capable of being separated from each other, so that the lateral row anchor is pushed out.

Optionally, the snap anchor housings have a snap protrusion, and the lateral row anchor has a snap slot fitting to the snap protrusion through insertion and connection.

Optionally, an inner side wall of the box body and a position corresponding to the snap anchor housings have a plurality of sliding slots extending along different directions, and an outer side wall of the snap anchor housings has a plurality of sliding rods slidably connected to the sliding slots, so that the end of the two snap anchor housings sleeved with the elastic sleeve is capable of being separated from or close to each other.

### BRIEF DESCRIPTION OF THE DRAWINGS

FIG. 1 is a schematic structural view of an implantation system for a lateral row anchor according to an embodiment of the present application;

FIG. 2 is a sectional structural view of an implantation system for a lateral row anchor according to an embodiment of the present application;

FIG. 3 is a schematic structural view of an implantation rod, a handle, and a protective sleeve in an implantation system for a lateral row anchor according to an embodiment of the present application;

FIG. 4 is an enlarged view of part A in an embodiment shown in FIG. 3;

FIG. 5 is a schematic structural view of a lateral row anchor in an implantation system for a lateral row anchor according to an embodiment of the present application;

FIG. 6 is a sectional structural view of a lateral row anchor in an implantation system for a lateral row anchor according to an embodiment of the present application;

FIG. 7 is a schematic structural view of an anchor box, an implantation rod, and a protective sleeve in an implantation system for a lateral row anchor according to an embodiment of the present application;

FIG. 8 is a schematic structural view of a lateral row anchor threaded with a suture in an implantation system for a lateral row anchor according to an embodiment of the present application;

FIG. 9 is a sectional structural view of clamp anchor components docked with each other in an implantation system for a lateral row anchor according to an embodiment of the present application;

FIG. 10 is a sectional structural view of clamp anchor components separated from each other in an implantation system for a lateral row anchor according to an embodiment of the present application; and

FIG. 11 is a schematic exploded structural view of clamp anchor components in an implantation system for a lateral row anchor according to an embodiment of the present application.

### REFERENCE NUMERALS

1: Snap head; 11: Fixation section; 12: Accommodation cavity; 13: Locking piece; 14: Abutting section; 15: Protective sleeve; 151: Guide block;
2: Implantation rod; 21: Handle; 211: Locking section; 22: Thread segment; 23: Connection segment; 24: Connection protrusion;
3: Anchor box; 31: Insertion channel; 32: Guide slot; 33: Box body; 34: Clamp anchor component; 341: Snap anchor housing; 342: Elastic sleeve; 343: Snap inclined surface 344: Snap protrusion; 345: Sliding rod; 35: Sliding slot;
4: Lateral row anchor; 41: Step hole; 42: Snap slot;
5: Suture;
6: Pull suture component; 61: Clip; 62: Metal ring.

### DETAILED DESCRIPTION

The present application will be described in further detail below with reference to the drawings and embodiments. It may be understood that specific embodiments described herein are merely intended to explain the present application and not to limit the present application. In addition, it should be noted that, for ease of description, only some, but not all, of structures related to the present application are shown in the drawings.

In the description of the present application, unless otherwise clearly specified and limited, the terms such as “linkage”, “connection”, “fixation” should be interpreted in a broad sense, for example, they may refer to a fixed connection, a detachable connection or integration; a mechanical connection, or an electrical connection; a direct connection, an indirect connection through an intermediate medium, or an internal connection or an interaction relationship between two elements. For those skilled in the art, the specific meanings of the above terms in the present application may be understood in accordance with specific conditions.

In the present application, unless otherwise clearly specified and limited, the first feature being “above” or “below” the second feature may include that the first feature and the second feature are in direct contact, or may include that the first feature and the second feature are not in direct contact but are in contact by another feature between the first feature and the second feature. Also, the first feature is “over”, “above” and “on” the second feature indicates that the first feature is directly above or diagonally above the second feature, or only indicates that the first feature is horizontally higher than the second feature. The first feature is “below”, “under” and “beneath” the second feature indicates the first feature is directly under or obliquely under the second feature, or only indicates that the first feature is horizontally lower than the second feature.

In the description of the embodiments, the orientation or positional relationship indicated by the terms such as “upper”, “lower”, “left”, “right” is based on the orientation or positional relationship shown in the drawings, and is only for facilitating the description and simplifying the operation, but not for indicating or implying that the referred apparatus or element must have a particular orientation, or be configured and operated in a particular orientation, which cannot be understood as the limitation to the present application. In addition, the terms “first” and “second” are merely used for distinguishing in description, and do not have a special meaning.

Embodiments of the present application disclose an implantation apparatus for a lateral row anchor and an implantation system for a lateral row anchor.

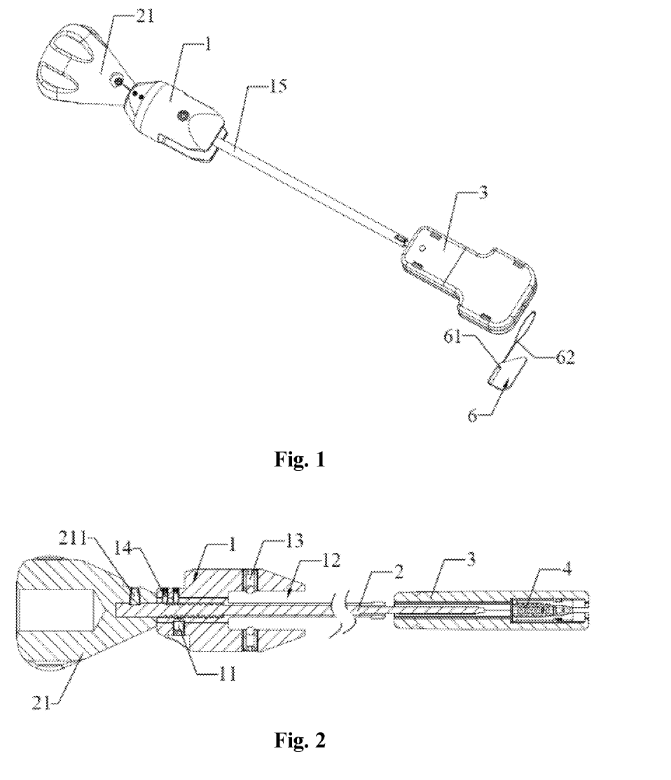

Referring to FIGS. 1-3, the implantation apparatus for a lateral row anchor includes the snap head 1, the implantation rod 2, and the handle 21. The snap head 1 has the connection channel; the implantation rod 2 passes through the connection channel, one end of the implantation rod 2 passing through the connection channel can be inserted to the anchor box 3 and is inserted and connected to the lateral row anchor 4 in the anchor box 3 for assembly, and configured to implant the lateral row anchor 4 threaded with the suture 5 into the designated position after the lateral row anchor 4 threaded with the suture 5 is ejected out of the anchor box 3; the handle 21 is provided at the other end of the implantation rod 2 and configured to drive the implantation rod 2 to rotate, the implantation rod 2 has the thread segment 22, and the snap head 1 is provided with the abutting section 14, the abutting section 14 is snapped into the thread of the thread segment 22 to drive the implantation rod 2 to move along the axial direction relative to the snap head 1 and implant the lateral row anchor 4 into the designated position under a condition that the implantation rod 2 rotates.

Specifically, the snap head 1 is a block in shape, the middle part of the snap head 1 is penetrated and provided with the via as the connection channel, the implantation rod 2 is a long strip in shape, one end of the implantation rod 2 passes through the connection channel and is inserted and connected to the anchor box 3, the lateral row anchor 4 is pre-stored in the anchor box 3, and one end of the implantation rod 2 inserted into the anchor box 3 is inserted and connected to the lateral row anchor 4 and fixed to the lateral row anchor 4 for assembly. One end of the implantation rod 2 away from the anchor box 3 is provided with the handle 21, the fixed connection between the handle 21 and the implantation rod 2 may be achieved by a detachable connection such as a snap-fitting or a thread connection, or by a non-detachable connection such as welding, bonding or integral forming.

The thread segment 22 is provided in the part of the implantation rod 2 located in the connection channel of the snap head 1, the thread in the thread segment 22 may be a trapezoid or a triangle in shape, and the guide slot is formed between adjacent two threads; the abutting section 14 is provided on the snap head 1, and the abutting section 14 can at least partially extend into the connection channel and be inserted to the guide slot, that is, the abutting section 14 and the thread form a snap fit. In this embodiment, the abutting section 14 is a screw, the snap head 1 is penetrated and provided with the thread hole communicated with the connection channel, the screw is threaded and connected to the thread hole, and the lower end of the screw is inserted and connected to the guide slot. In other embodiments, the abutting section 14 may be a snap rod; under this condition, only the via needs to be provided on the snap head 1, so that one end of the snap rod can be inserted to the connection channel, then the via is closed by a structure such as a block to lock the snap rod. Specifically, the implementation of the abutting section 14 is not limited by the present application as long as the abutting section 14 and the thread of the thread segment 22 can form a snap fit.

During assembly, one end of the implantation rod 2 pass through the connection channel, and one end of the implantation rod 2 passing through the connection channel is inserted and connected to the anchor box 3, so that the implantation rod 2 is inserted to the lateral row anchor 4, and the implantation rod 2 and the lateral row anchor 4 form a fixed insertion and connection. Under this condition, the lateral row anchor 4 may be threaded with a suture to pass the suture 5 through the lateral row anchor 4 and continue to push the implantation rod 2, so that implantation rod 2 squeezes the lateral row anchor 4 until the lateral row anchor 4 is pushed out of the anchor box 3. During implantation, the handle 21 is grasped to drive the implantation rod 2 to rotate, and the abutting section 14 closely abuts against the thread in the thread segment 22, so that the implantation rod 2 moves along the axial direction relative to the snap head 1 during the rotation and implants the lateral row anchor 4 into the designated position gradually. After the implantation is completed, the implantation rod 2 is separated from the lateral row anchor 4, then the anchor box 3 is withdrawn from the implantation rod 2. The anchor box 3 may be discarded, and the implantation apparatus may be recycled. Therefore, when the implantation apparatus for a lateral row anchor is used, in an aspect, the lateral row anchor 4 is stored in the anchor box 3, and the implantation rod 2 and the anchor box 3 are provided in a form of housing insertion and connection assembly, so that the implantation rod 2 and the anchor box 3 form a detachable connection, and after the lateral row anchor 4 is implanted, the implantation apparatus can be recycled to improve the utilization rate of the implantation apparatus and reduce the waste of medical resources; in another aspect, during the implantation process of the lateral row anchor 4, forming the dynamic spiral coupling by fitting the abutting section 14 to the thread segment 22 through thread can prevent the problem that the traditional thread connection easily generates the stuck and the wear during the transmission process and reduce the contact area of the thread, so that the vibration and the noise during the transmission process are reduced, the accuracy of the axial displacement control is improved, and the stability and the reliability of the implantation process are improved, which is conducive to achieving the high accuracy implantation of the lateral row anchor 4 during the operation.

Optionally, a plurality of abutting sections 14 are provided at intervals.

Specifically, the plurality of abutting sections 14 may be distributed along the extending direction of the connection channel, and the plurality of abutting sections 14 may be symmetrically arranged to improve the mechanical equilibrium during the transmission process, so that the smoothness and the durability of the implantation rod 2 during the rotation are further improved.

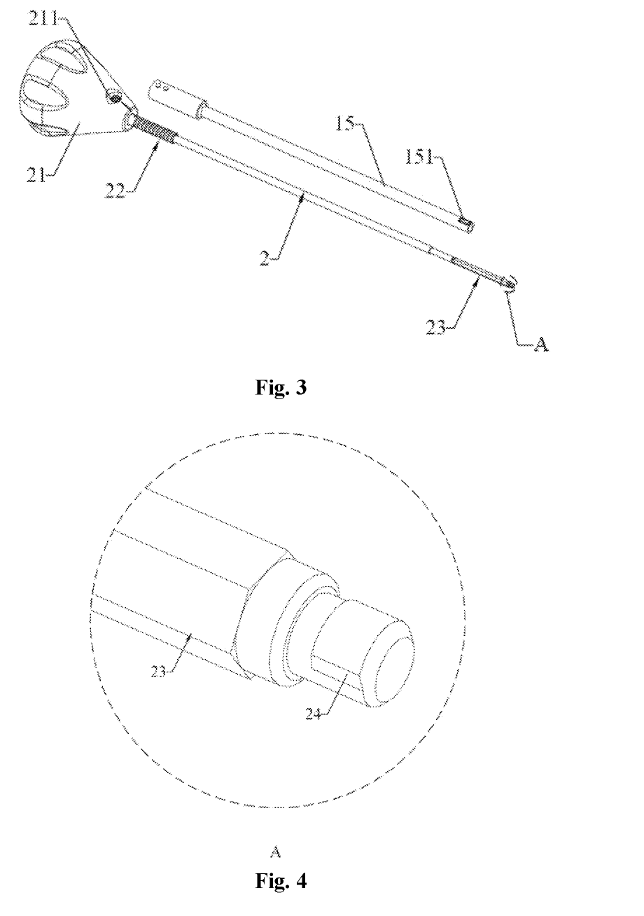

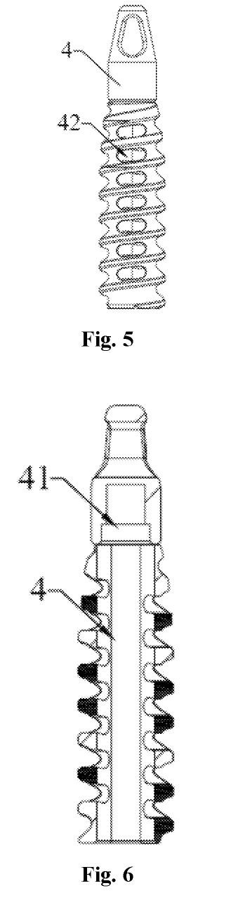

Referring to FIGS. 3-6, optionally, an end of the implantation rod 2 away from the handle 21 has the connection segment 23 that can be inserted to the lateral row anchor 4, and the head end of the connection segment 23 has the connection protrusion 24 for closely abutting against the inner wall of the anchor head of the lateral row anchor 4.

Specifically, the head end of the connection segment 23 may be a stair in shape, the connection protrusion 24 may be provided on the small diameter surface of the connection segment 23, and the connection protrusion 24 may have an inclined surface, a cambered surface, a spherical surface, or the like which can closely abut against the inner wall of the anchor head to form an interference fit when the anchor head of the lateral row anchor 4 is inserted.

Optionally, the connection protrusion 24 is an arc in shape, and the inner side of the anchor head of the lateral row anchor 4 has the step hole 41 closely abutting against the connection protrusion 24.

Specifically, the step hole 41 is provided on the inner side of the anchor head of the lateral row anchor 4, and the bore diameter is reduced along the direction in which the connection segment 23 is inserted, so that the arc surface of the connection protrusion 24 can closely abut against the hole wall of the small-diameter hole in the step hole 41 to form an interference fit.

The lateral row anchor 4 and the connection segment 23 are fixed by forming the interference fit between the connection segment 23 and the lateral row anchor 4, so that the radial compression is formed between the connection protrusion 24 and the lateral row anchor 4, and the head of the lateral row anchor 4 is smoothly driven to rotate synchronously when the implantation rod 2 is rotated; compared to the thread connection, the interference fit formed by the radial extrusion of the cambered surface can increase the contact area between the connection segment 23 and the lateral row anchor 4, so that more stable force transmission and greater connection strength are achieved to further ensure the high accuracy implantation of the lateral row anchor 4.

Optionally, the section of the connection segment 23 is a polygon in shape, and the side wall of the connection segment 23 is fitted to the inner side wall of the lateral row anchor 4.

Specifically, the section of the connection segment 23 may be a polygon in shape to adapt to the anchor body of the lateral row anchor 4, and the shape of the specific connection segment 23 may be designed based on the anchor body of the lateral row anchor 4, which is not limited by the present application. The section of the connection segment 23 is a polygon in shape, so that a plurality of surface contacts are formed between the connection segment 23 and the anchor body of the lateral row anchor 4 to ensure that the lateral row anchor 4 and the implantation rod 2 can rotate synchronously.

Referring to FIG. 2, optionally, the implantation rod 2 is inserted and connected to the handle 21, the handle 21 is provided with the locking section 211, and the locking section 211 abuts against an end of the implantation rod 2 inserted and connected to the handle 21.

Specifically, the front end of the handle 21 is provided with the blind via, the implantation rod 2 is inserted to the blind via, the locking section 211 is inserted to the side wall of the handle 21, and the locking section 211 can extend into the blind via and closely abut against the side wall of the implantation rod 2, so that the implantation rod 2 is fixed in the blind via. The locking section 211 may be a screw, and the handle 21 is correspondingly provided with the thread hole. The locking section 211 may also be an insertion rod, under this condition, the handle 21 only needs to be correspondingly provided with a via communicated with the blind via, and a snap hole may be correspondingly provided on the implantation rod 2 for being inserted by the insertion rod. The specific structure of the locking section 211 can be designed based on the actual assembly difficulty, which is not limited by the present application.

The handle 21 and the implantation rod 2 are in the detachable connection, so that the replacement of any structure can be convenient, the utilization rate of the structure is improved, and the waste of medical resources is reduced; at the same time, the fixation of the handle 21 and the implantation rod 2 can be achieved by the simple fitting through insertion and connection, so that the assembly process is simple and easy to operate.

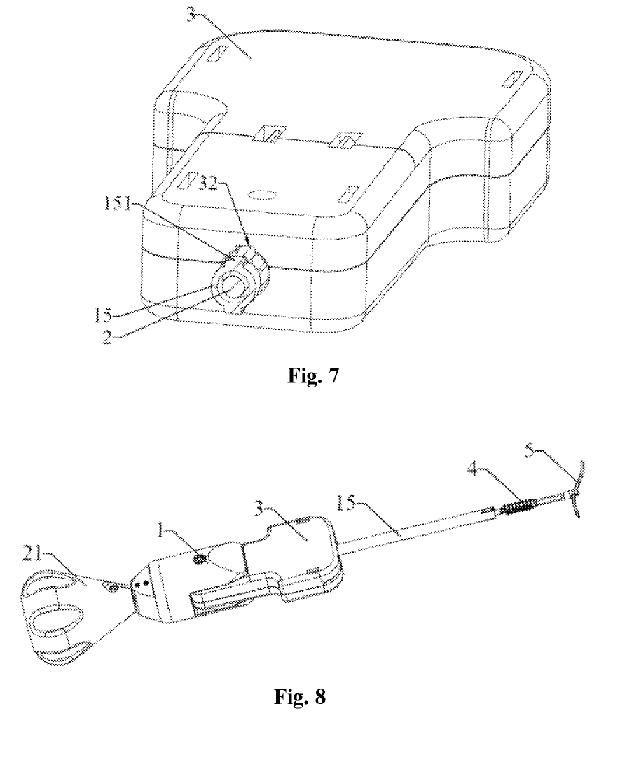

Referring to FIGS. 2, 3 and 7, optionally, the implantation apparatus for a lateral row anchor further includes the protective sleeve 15. The protective sleeve 15 is sleeved on the thread segment 22 of the implantation rod 2, and the abutting section 14 passes through the protective sleeve 15 and is snapped into the thread of the thread segment 22.

Specifically, one end of the protective sleeve 15 is fixedly connected to the snap head 1, and the protective sleeve 15 and the snap head 1 may be an integral structure or may be fixed together by bonding or welding. The other end of the protective sleeve 15 passes through the connection channel, the implantation rod 2 is located in the protective sleeve 15, and the inner diameter of the protective sleeve 15 is greater than the diameter of the implantation rod 2, so that a movable gap is formed between the protective sleeve 15 and the implantation rod 2 to ensure that the implantation rod 2 can smoothly rotate. The position where the protective sleeve 15 corresponds to the abutting section 14 is provided with the via for being inserted by the abutting section 14. The protective sleeve 15 covers the entire thread segment 22 and extends to a side close to the connection segment 23 along the axial direction of the implantation rod 2, and can also be inserted and connected to the anchor box 3.

The protective sleeve 15 is provided, so that, in an aspect, the protective sleeve 15 can serve the function of guiding during the insertion process to ensure that the implantation rod 2 does not deflect; in another aspect, the protective sleeve 15 can serve the function of protecting the relatively long implantation rod 2 and prevent the implantation rod 2 from being damaged.

Optionally, the snap head 1 has the fixation section 11, and the fixation section 11 abuts against the outer side wall of the protective sleeve 15.

Specifically, the protective sleeve 15 is fitted to the connection channel through insertion and connection, the fixation section 11 is provided through the snap head 1, the fixation section 11 partially extends into the connection channel, the fixation slot is provided on the outer side wall of the protective sleeve 15, and the fixation section 11 can closely abut against the slot bottom wall of the fixation slot, so that the protective sleeve 15 is fixed on the snap head 1. In this embodiment, the fixation section 11 is a screw; and in other embodiments, the fixation section 11 may be a structure such as an insertion and connection rod as long as the fixation section 11 can fix the protective sleeve 15.

The protective sleeve 15 and the snap head 1 are in the detachable connection, so that the protective sleeve 15 may be separated from the snap head 1 to facilitate the replacement of either one of the protective sleeve 15 or the snap head 1 and further save the medical resources.

Optionally, the protective sleeve 15 has the guide block 151, the anchor box 3 has the insertion channel 31 to which the protective sleeve 15 is inserted, and the insertion channel 31 has the guide slot 32 on which the guide block 151 slides.

Specifically, the insertion channel 31 is provided in the anchor box 3, the insertion channel 31 is communicated with the lateral row anchor 4, the guide slot 32 is provided on the side wall of the insertion channel 31, the guide slot 32 extends along the length direction of the insertion channel 31, and the guide block 151 is adapted to the guide slot 32. In this embodiment, each of the upper side and the lower side of the protective sleeve 15 is provided with the guide block 151 in cuboid shape; correspondingly, one guide slot 32 is provided on each of the upper side and the lower side of the insertion channel 31.

The guide block 151 and the guide slot 32 are provided, so that when the guide block 151 and the guide slot 32 are fitted to the anchor box 3 through insertion and connection, fitting the guide block 151 to the guide slot 32 can accurately define the moving direction to ensure that the implantation rod 2 is accurately inserted to the lateral row anchor 4.

Optionally, the snap head 1 has the accommodation cavity 12 for accommodating the anchor box 3, and the cavity wall of the accommodation cavity 12 is provided with the locking piece 13 for snapping into the anchor box 3 to lock the anchor box 3 in the accommodation cavity 12.

Specifically, an end of the snap head 1 away from the handle 21 is in U-shape to form the accommodation cavity 12, the locking piece 13 is provided on the cavity wall of the accommodation cavity 12, and the locking piece 13 has certain elasticity, so that the locking piece 13 can contract when the anchor box 3 is inserted and release after the anchor box 3 is inserted, so as to form a snap fixation with the anchor box 3. In this embodiment, the locking piece 13 is a spring plunger, and the side wall of the anchor box 3 is provided with a snap hole snap-fitting to the anchor box 3. In order to improve the snapping effect, two locking pieces 13 opposite to each other are provided and located on the upper side and the lower side of the anchor box 3, respectively.

When the lateral row anchor 4 is pushed out of the anchor box 3, the anchor box 3 can be inserted and connected to the accommodation cavity 12, and the anchor box 3 and the locking piece 13 form a snap fit, so that the anchor box 3 and the snap head 1 form the fixed connection.

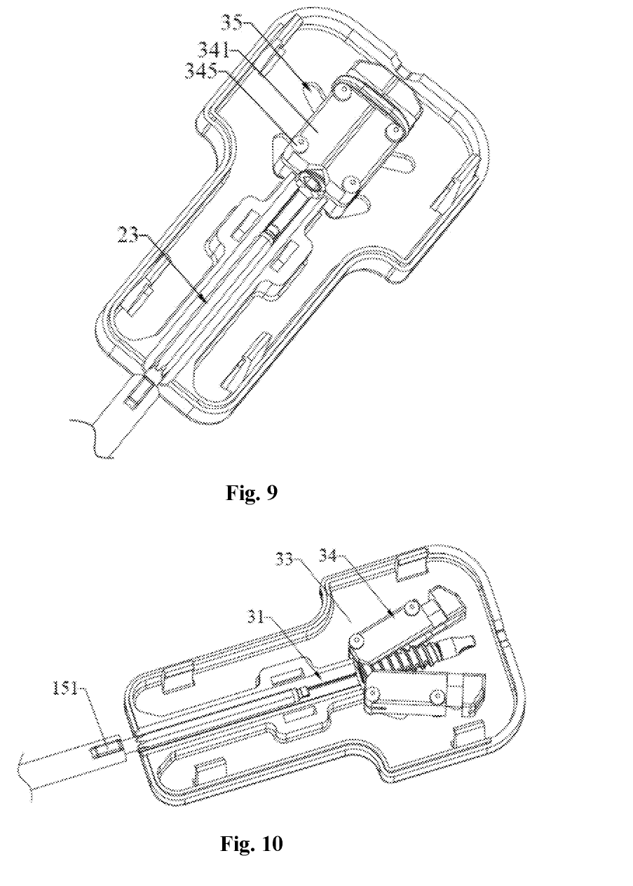

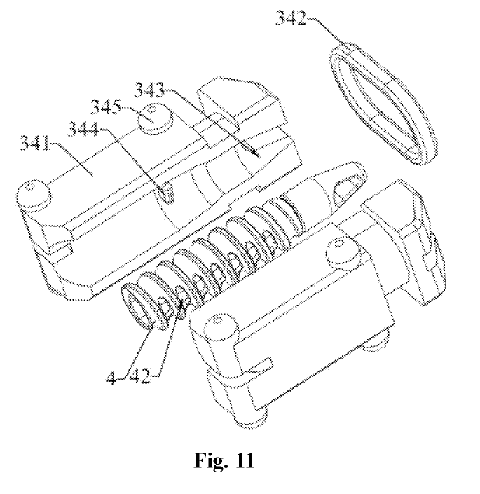

Referring to FIGS. 8-11, the implantation system for a lateral row anchor includes the anchor box 3, the pull suture component 6 (referring to FIG. 1), and the implantation apparatus for a lateral row anchor in the above embodiments. The anchor box 3 stores the lateral row anchor 4 and is fitted to the implantation apparatus for a lateral row anchor through insertion and connection; and the pull suture component 6 is configured to thread the suture 5 for the lateral row anchor 4 in the anchor box 3.

When the implantation system is used, first the implantation apparatus for a lateral row anchor is inserted and connected to the lateral row anchor 4 stored in the anchor box 3 for assembly, then the suture 5 is threaded for the lateral row anchor 4 using the pull suture component 6, and after the suture 5 passes through the lateral row anchor 4, the lateral row anchor 4 is pushed out of the anchor box 3 by the implantation apparatus to complete the assembly of the implantation apparatus and the lateral row anchor. After the implantation is completed, the implantation apparatus may be separated from the anchor box 3, and the implantation apparatus may be recycled to reduce the waste of medical resources.

Optionally, the pull suture component 6 (referring to FIG. 1) includes the clip 61 and the metal ring 62 provided on the clip 61, the suture 5 is threaded in the metal ring 62, and the metal ring 62 is driven to move by the clip 61, so that the suture 5 is brought out of the anchor box 3 after the suture 5 passes through the lateral row anchor 4.

Optionally, the anchor box 3 includes the box body 33 and the clamp anchor component 34. One end of the box body 33 has the insertion channel 31 to which the implantation rod 2 is partially inserted, and the other end has the anchor-discharging channel for the lateral row anchor 4 to slide out; the clamp anchor component 34 is provided in the box body 33 and correspondingly communicated with the insertion channel 31, and the lateral row anchor 4 is clamped in the clamp anchor component 34.

Specifically, the anchor box 3 is a long strip in shape and is formed by snapping of the upper housing body and the lower housing body, the insertion channel 31 and a space for accommodating the clamp anchor component 34 are formed between the upper housing body and the lower housing body, the clamp anchor component 34 is communicated with the insertion channel 31, a side of the box body 33 away from the insertion channel 31 is penetrated and provided with the anchor-discharging channel, and the anchor-discharging channel corresponds to the clamp anchor component 34, so that the lateral row anchor 4 can be discharged through the anchor-discharging channel; the side surface of the box body 33 is provided with a notch for the suture 5 to pass through the box body 33 and thread into the lateral row anchor 4.

Optionally, the clamp anchor component 34 includes two snap anchor housings 341 opposite to each other, the lateral row anchor 4 is stored between the two snap anchor housings 341, the snap anchor housings 341 are actively connected to the box body 33, one end of the snap anchor housings 341 away from the insertion channel 31 is sleeved with the elastic sleeve 342, the inner side and one end away from the insertion channel 31 of the snap anchor housings 341 have the snap inclined surface 343, the snap inclined surface 343 abuts against the head end of the lateral row anchor 4, and the one end of the two snap anchor housings 341 sleeved with the elastic sleeve 342 is separate from each other, so that the lateral row anchor 4 is pushed out.

Specifically, the snap anchor housing 341 is a long strip in shape and hollow inside, and under a condition that the two snap anchor housings 341 are assembled together, the storage cavity for accommodating the lateral row anchor 4 is formed. The lateral row anchor 4 includes the main body and the anchor head, a part of the snap anchor housing 341 close to the anchor head is provided with the snap inclined surface 343, and the snap inclined surface 343 can be adapted to the inclined surface of the anchor head, so that the lateral row anchor 4 and the snap anchor housing 341 form a surface contact. In this embodiment, the inclined angle of the snap inclined surface 343 may be between 0° and 90°. The suture 5 is threaded to the threading hole of the anchor head, and the snap anchor housing 341 has a notch corresponding to and communicating with the threading hole.

One end of the snap anchor housing 341 close to the anchor head is sleeved with the elastic sleeve 342, the elastic sleeve 342 may be made of a silica gel material or may be made of other materials, which is not limited by the present application. The snap anchor housings 341 themselves may be fitted to each other by slot holes, and the separation of the two snap anchor housings 341 from each other can be achieved in the box body 33, or the two snap anchor housings 341 can adjust one end of the elastic sleeve 342 to be separated from each other by a hinging structure as long as the lateral row anchor 4 can be smoothly discharged.

The lateral row anchor 4 is limited in the box body 33 by the two snap anchor housings 341, and the snap inclined surface 343 is provided, so that the lateral row anchor and the snap housing form a snap fit and are fitted to the elastic sleeve 342 to assemble the two snap anchor housings 341 together, and it is ensured that the lateral row anchor 4 is correspondingly communicated with the insertion channel 31; after the implantation rod 2 is inserted into the lateral row anchor 4 through the insertion channel 31, the implantation rod 2 can be smoothly inserted to the lateral row anchor 4 and push the lateral row anchor 4 to move, the anchor head abuts against the snap inclined surface 343 to push the two snap anchor housings 341 to separate from each other, and the lateral row anchor 4 can be smoothly pushed out of the anchor box 3.

Optionally, the snap anchor housings 341 have the snap protrusion 344, and the lateral row anchor 4 has the snap slot 42 fitting to the snap protrusion 344 through insertion and connection.

Specifically, the main body of the lateral row anchor 4 is provided with a plurality of snap slots 42, the inner side wall of the snap anchor housing 341 is provided with the snap protrusion 344, and the snap protrusion 344 may be a polygon in shape, such as a quadrilateral column in shape or a pentagonal column in shape, and the corresponding snap slot 42 is also a polygon in shape.

The snap protrusion 344 and the snap slot 42 are provided, so that the lateral row anchor 4 is provided in two snap housings, the anchor head can be limited by the snap inclined surface 343, and the main body forms a limit by fitting the snap protrusion 344 to the snap slot 42 through insertion and connection, so that the clamp anchor component 34 can limit the anchor head and the main body of the lateral row anchor 4 at the same time, effectively improving the limit effect of the lateral row anchor 4.

Optionally, the inner side wall of the box body 33 and the position corresponding to the snap anchor housings 341 have a plurality of sliding slots 35 extending along different directions, and the outer side wall of the snap anchor housings 341 has a plurality of sliding rods 345 slidably connected to the sliding slots 35, so that one end of the two snap anchor housings 341 sleeved with the elastic sleeve 342 can be separated from or close to each other.

Specifically, each snap anchor housing 341 is correspondingly provided with four sliding rods 345, two of the sliding rods 345 are provided on the top wall of the snap anchor housing 341, two of the sliding rods 345 are provided on the bottom wall, and the inner top wall and the inner bottom wall of the box body 33 are correspondingly provided with the sliding slots 35. The top wall may be taken as an example for illustrating the distribution of the specific sliding rods 345, one of the sliding rods 345 is provided close to the insertion channel 31, and the other of the sliding rods 345 is provided close to the elastic sleeve 342. The slide rods 345 on the bottom wall are provided in the same manner.

The extending direction of the sliding slots 35 is referred to the extending direction of the insertion channel 31, the included angle between the sliding slot 35 close to the insertion channel 31 and the extending direction is relatively small, and the included angle between the sliding slot 35 away from the insertion channel 31 and the extending direction is relatively great, so that the one end of the two opposite snap anchor housings 341 sleeved with the elastic sleeve 342 can be separated from each other, and the magnitude of the specific included angle may be designed based on the actual dimension of the lateral row anchor 4 as long as it can be ensured that the lateral row anchor 4 is smoothly pushed out.

The separation of the snap anchor housings 341 is achieved by providing the sliding rods 345 and the sliding slots 35, so that the clamp anchor component 34 does not need to be provided with other structures such as a hinge during assembly, the overall structural complexity can be effectively reduced, and the assembly difficulty and the manufacturing difficulty can be reduced.

Apparently, the above embodiments of the present application are merely for the purpose of clearly illustrating the examples given in the present application, and are not intended to limit the embodiments of the present application. For those skilled in the art, various obvious changes, readjustments, and replacements can be made without departing from the protection scope of the present application. All embodiments need not and cannot be exhaustive herein. Any modification, equivalent replacement, improvement, and the like made within the gist and principle of the present application shall be should be included in the protection scope of the claims of the present application.

What is claimed is:

1. An implantation apparatus for a lateral row anchor comprising:
a snap head (1) having a connection channel;
an implantation rod (2) passing through the connection channel, wherein an end of the implantation rod (2) passing through the connection channel is capable of being inserted to an anchor box (3) and inserted and connected to a lateral row anchor (4) in the anchor box (3) and is configured to implant the lateral row anchor (4) threaded with a suture (5) into a designated position after the lateral row anchor (4) threaded with the suture (5) is ejected out of the anchor box (3);
a handle (21) provided at the other end of the implantation rod (2) and configured to drive the implantation rod (2) to rotate, wherein the implantation rod (2) has a thread segment (22), and the snap head (1) is provided with an abutting section (14), the abutting section (14) at least partially extend into the connection channel and is snapped into a thread of the thread segment (22) to form a dynamic spiral coupling and reduce a contact area of the thread, so as to drive, under a condition that the implantation rod (2) rotates, the implantation rod (2) to move along an axial direction relative to the snap head (1) and implant the lateral row anchor (4) into the designated position; and
a protective sleeve (15) sleeved on the thread segment (22) of the implantation rod (2), wherein the abutting section (14) passes through the protective sleeve (15) and is snapped into the thread of the thread segment (22).

2. The apparatus according to claim 1, wherein a plurality of the abutting sections (14) are provided at intervals.

3. The apparatus according to claim 1, wherein an end of the implantation rod (2) away from the handle (21) has a connection segment (23) capable of being inserted to the lateral row anchor (4), and a head end of the connection segment (23) has a connection protrusion (24) for closely abutting against an inner wall of the lateral row anchor (4).

4. The apparatus according to claim 3, wherein the connection protrusion (24) is an arc in shape, and an inner side of a head of the lateral row anchor (4) has a step hole (41) closely abutting against the connection protrusion (24).

5. The apparatus according to claim 3, wherein a section of the connection segment (23) is a polygon in shape, and a side wall of the connection segment (23) is fitted to an inner side wall of the lateral row anchor (4).

6. The apparatus according to claim 1, wherein the implantation rod (2) is inserted and connected to the handle (21), and the handle (21) is provided with a locking section (211) abutting against an end of the implantation rod (2) inserted and connected to the handle (21).

7. The apparatus according to claim 1, wherein the snap head (1) has a fixation section (11) abutting against an outer side wall of the protective sleeve (15).

8. The apparatus according to claim 1, wherein the protective sleeve (15) has a guide block (151), the anchor box (3) has an insertion channel (31) to which the protective sleeve (15) is inserted, and the insertion channel (31) has a guide slot (32) on which the guide block (151) slides.

9. The apparatus according to claim 1, wherein the snap head (1) has an accommodation cavity (12) for accommodating the anchor box (3), and a cavity wall of the accommodation cavity (12) is provided with a locking piece (13) for snapping into the anchor box (3) to lock the anchor box (3) in the accommodation cavity (12).

10. An implantation system for a lateral row anchor comprising:
the implantation apparatus for the lateral row anchor according to claim 1;
the anchor box (3) storing the lateral row anchor (4) and inserted and connected to the implantation apparatus for the lateral row anchor; and
a pull suture component (6) for threading the suture (5) for the lateral row anchor (4) in the anchor box (3).

11. The system according to claim 10, wherein the anchor box (3) comprises:
a box body (33), wherein an end of the box body (33) has an insertion channel (31) to which the implantation rod (2) is partially inserted, and the other end has an anchor-discharging channel for the lateral row anchor (4) to slide out; and
a clamp anchor component (34) provided in the box body (33) and correspondingly communicated with the insertion channel (31), wherein the lateral row anchor (4) is clamped in the clamp anchor component (34).

12. The system according to claim 11, wherein the clamp anchor component (34) comprises two snap anchor housings (341) opposite to each other, the lateral row anchor (4) is stored between two of the snap anchor housings (341), the snap anchor housings (341) are actively connected to the box body (33), an end of the snap anchor housings (341) away from the insertion channel (31) is sleeved with an elastic sleeve (342), an inner side and an end away from the insertion channel (31) of the snap anchor housings (341) have a snap inclined surface (343) abutting against a head end of the lateral row anchor (4), and the end of the two of the snap anchor housings (341) sleeved with the elastic sleeve (342) is capable of being separated from each other, so that the lateral row anchor (4) is pushed out.

13. The implantation system according to claim 12, wherein the snap anchor housings (341) have a snap protrusion (344), and the lateral row anchor (4) has a snap slot (42) fitting to the snap protrusion (344) through insertion and connection.

14. The implantation system according to claim 12, wherein an inner side wall of the box body (33) and a position corresponding to the snap anchor housings (341) have a plurality of sliding slots (35) extending along different directions, and an outer side wall of the snap anchor housings (341) has a plurality of sliding rods (345) slidably connected to the sliding slots (35), so that the end of the two of the snap anchor housings (341) sleeved with the elastic sleeve (342) is capable of being separated from or close to each other.
